(19) United States
(12) Patent Application Publication
(10) Pub. No.: US 2004/0063167 A1
(43) Pub. Date: Apr. 1, 2004

(54) Minimising calibration problems of in vivo glucose sensors

(76) Inventors: Peter Kaastrup, Maaloev (DK); Mads Christian Sabra, Copenhagen N (DK)

Correspondence Address: Reza Green, Esq., Novo Nordisk Pharmaceuticals, Inc., 100 College Road West, Princeton, NJ, 08540, US

(21) Appl. No.: 10/617,617

(22) Filed: Jul. 11, 2003

(30) Foreign Application Priority Data
Jul. 12, 2002 (DK) PA 2002 01103

(51) Int. Cl. C12Q 1/54; C12Q 1/30

(52) U.S. Cl. 435/27; 435/14

(57) ABSTRACT

The present invention pertains to the minimisation of calibration problems of glucose sensors. Accordingly, the invention provides a method of improving the performance of a ROS producing glucose sensor, said method comprising providing the glucose sensor with a ROS removing compartment capable of reducing the diffusion of ROS out of the glucose sensor to a level at which biointerference is abolished or substantially reduced. The invention further relates to use of a ROS removing compartment in a ROS producing glucose, a ROS producing glucose sensor comprising a ROS removing compartment, and to the use of such a sensor in a human.

### FIELD OF INVENTION

Implanted or semi-implanted glucose sensors for monitoring of blood glucose in the regulation of e.g. diabetes mellitus are well known. However, it is a significant problem that the sensors presently available do not function adequately in vivo over a sufficient time period in spite of the fact that the sensors function well in vitro. Hence, for state of the art sensors it is necessary to calibrate the sensors a number of times e.g. at least four times daily because the sensitivity of the sensor changes over time. Although a number of reasons for this problem have been suggested, none of the proposed solutions have been able to solve the problem and the reason for the sensitivity problem is still unknown.

The present invention relates to the finding that by using a glucose sensor with an outer membrane comprising catalase and/or other reactive oxygen species scavengers, in order to secure that reactive oxygen species do not diffuse out of the sensor to the surroundings, the calibration problems are significantly reduced. It is important that the reactive oxygen species is reduced to a concentration much lower than the concentration where it will exert cytotoxic effects.

The present invention thus relates to a method of improving the performance of a ROS producing glucose sensor, said method comprising providing the glucose sensor with a ROS removing compartment capable of reducing the diffusion of ROS out of the glucose sensor to a level at which biointerference is abolished or substantially reduced. The invention further relates to use of a ROS removing compartment in a ROS producing glucose, a ROS producing glucose sensor comprising a ROS removing compartment, and to the use of such a sensor in a human.

### BACKGROUND OF THE INVENTION

In some way biosensor implants represent an extreme variant of (xeno)transplantation and a lot of relevant models of tissue interactions and relevant experimental data can be drawn from basic immunological studies.

Immunology has been defined as the science of self-nonself discrimination. Slightly altered conformation of the major plasma/lymph proteins at the surface of the implant may be an initial trigger of immunological responses.

Nonself does not necessary trigger a strong response. A “danger” signal is often also required. The danger signal may simply result from mechanical disruption of a few cells or capillary vessels and the release of cell membrane or tissue fragments. Besides immunology, the literature related to wound healing and tissue repair is therefore also very relevant.

On a time scale the basic tissue interactions with an implant (or transplant) are:

Immediately/short term (seconds to hours)

Disruption of cells, release of cell fragments “danger” signals (=>clot formation, coagulation factors, complement factors, mobilisation of different inflammatory cell types, release of cytokine subsets. Start of immune and repair processes

Intermediate (hours to several days)

Clot resolution, mobitisation of new subsets of different cell types as macrophages and fibroblast, release of new cytokine subsets, extracellulary protein matrix deposition, wound healing, adapted/recombinatory immune response, eventually a rejection process starts.

Chronically Stage

Scar formation processes (active for ever), memory formation of immune response

Encapsulation

The processes outlined above contribute to the above-discussed problems of having biosensors to function adequately in vivo. A useful review of implanted electrochemical glucose sensors for the management of diabetes can be found in Heller et al., 1999.

One State of the art manufacturer (Minimed Inc.) of biosensors has several patents, and of these drawings of sensor geometry and coatings can be found in e.g. U.S. Ser. No. 2001/0,008,931.

In the function of most glucose sensors based upon glucose oxidase, H₂O₂ is produced continuously. Hydrogen peroxide appears to be a ubiquitous molecule. Multiple papers have described high (usually ≧50 μM) levels of H₂O₂ as being cytotoxic to a wide range of animal, plant and bacterial cells in culture. However, levels of H₂O₂ at or below about 20-50 μM seem to have limited cytotoxicity to many cell types (Halliwell et al., 2000).

WO94/10560 describes a glucose oxidase sensor with a catalase membrane which regenerates a part of the oxygen consumed by the glucose oxidation in order to improve the performance of the glucose sensor by making the regenerated oxygen available to the enzymatic reaction of the glucose oxidase.

None of these references deal with the problem of the present invention which is to reduce H₂O₂ to a concentration much lower than the concentration where it will exert cytotoxic effects as described in detail in the following.

### DETAILED DESCRIPTION OF THE INVENTION

The present invention relates to glucose sensors producing reactive oxygen species. Examples of ROS producing glucose sensors are glucose oxidase based glucose sensors which generate and release H₂O₂ to their surroundings when functioning.

In general terms such an electrochemical sensor according to the invention will comprise a working electrode which comprises the following:

1. glucose oxidase producing H₂O₂ and an H₂O₂ detecting electrode (central part)

2. diffusion compartment through which glucose and O₂ diffuse to the glucose oxidase and the electrode and excess H₂O₂ may diffuse out to the body

3. ROS removing compartment which may comprise catalase or other reactive oxygen species scavenger. It has a membrane function in that glucose, O₂ and other low molecular weight substances diffuse to the glucose oxidase and the electrode. Another important function of the compartment is to avoid that excess H₂O₂ diffuses out to the body as described in detail in the following.

4. Semipermeable compartment which hinders access of cells and high molecular compounds to the central part but allowing access for molecules having a molecular weight of less than e.g. 500D.

5. Biocompatible compartment providing the interface between the sensor and the body having properties to avoid membrane biofouling

3 and 4 may be the same compartment, 4 and 5 may be the same compartment, and 3, 4 and 5 may be the same compartment.

The primary object of the present invention is to provide means for assuring that the glucose sensor functions adequately. In the present context, the sensor functions adequately when there is a significant correlation between physiological relevant glucose concentrations and the signal from the sensor.

The term ‘monocyte chemotaxis’ designates the processes by which a monocyte orients itself in a specific spatial relationship to a chemical stimulus. Monocyte chemotaxis may thus result in attraction and direction to the sites of various chemical substances.

Biofouling has been described as the adhesion of proteins and other biological matter on the surfaces of a sensor and causing decreased sensor signal. Membrane biofouling is a process that starts immediately upon contact of the sensor with the body when cells, proteins and other biological components adhere to the surface, and in some cases, impregnate the pores of the material. The membrane biofouling of the sensors outer membrane does impede analyte diffusion causing decreased sensor signal and it is believed that the adhering proteins are one of the main factors to modulate the longer term cellular and/or encapsulation process. Electrode fouling (electrode passivation) is a process that occurs on the interior of the sensor when substances from the body are able to penetrate the outer membranes and alter the electrode surface and causing decreased sensor signal (Wisniewski et al., 2000).

In the present context biointerference is defined as the processes which disturb the sensor signals executed around, on or in a sensor by the biological components of the body. The processes lead to altered diffusion conditions around the sensor caused by accumulation of cells or fouling of one or more, possibly all three types mentioned above.

The term ‘encapsulation’ is defined as an in vivo process in which fibroblasts, fibrocytes, collagen, and giant cells provide adherent, impermeable, avascular barriers around or enclosing implants.

ROS (including H₂O₂, O₂.⁻ and OH.) are important chemical mediators in the regulation of signal transduction processes involved in cell growth and differentiation (Sauer et al. 2001). As example H₂O₂ induces activation of the interleukin-6 promoter activating nuclear factor-κB through NFκ-B inducing kinase (Zhang et al., 2001).

A first aspect of the present invention thus relates to a method of improving the performance of a ROS producing glucose sensor, said method comprising providing the glucose sensor with a ROS removing compartment capable of reducing the diffusion of ROS out of the glucose sensor to a level at which biointerference is abolished or substantially reduced.

Another aspect of the invention relates to the use of a ROS removing compartment in a ROS producing glucose sensor so that biointerference is substantially decreased or avoided. Yet another aspect of the invention relates to a ROS producing glucose sensor comprising a ROS removing compartment capable of reducing the diffusion of ROS out of the glucose sensor to a level at which biointerference is abolished or substantially reduced. Finally, one aspect of the invention relates to the use of such a sensor in a human. As stated above, examples of ROS producing glucose sensors are glucose oxidase based glucose sensors.

It is to be understood that the following description of features and embodiments of the invention relates to all the above mentioned aspects of the invention.

TGFβ is a major local up-regulator of the extracellular matrix proteins in fibrosis. It also induces monocyte chemotaxis. TGFβ is activated by Reactive Oxygen Species (ROS). ROS are generated by reduction-oxidation reactions.

It is an object of the present invention to reduce the diffusion of ROSfrom a ROS producing glucose sensor, such as a glucose sensor based upon glucose oxidase, to a level where substantially no activation of TGFβ and substantially no monocyte chemotaxis occur. In preferred embodiments of the invention, the ROS is H₂O₂.

None of the prior art references have dealt with the problem of avoiding initiating the ROS cascade. In order to do to it, it is necessary to reduce the level of e.g. hydrogen peroxide to a level which is significantly lower than previously considered, i.e. to a level which is significantly lower than the level which has previous been considered safe, i.e. considerably lower than 20 μM. In preferred embodiments of the invention the diffusion of ROS, such as H₂O₂, out of the sensor is reduced so that the concentration in the tissue surrounding the glucose sensor remains below 10 μM.

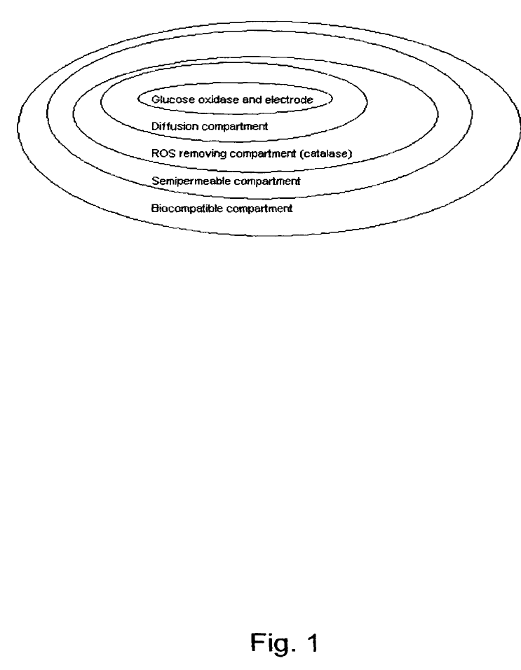

The method of the invention may be accomplished by introducing in a ROS producing glucose sensor, such as a glucose sensor based on glucose oxidase electrodes, a specially placed and specially composed compartment in the glucose sensor, which will minimise release of H₂O₂ and the related undesired tissue interaction and attraction of inflammatory cells. The compartment surrounds the electrode and may contain catalase and/or one or more other reactive oxygen species scavengers for removing ROS, such as hydrogen peroxide, and their reactive oxidative decay products, and may be placed inside semipermeable and biocompatible outer compartments (see FIG. 1).

Placing catalase and/or one or more other reactive oxygen species scavengers in a semipermeable compartment placed between the ROS producing electrode compartment and body tissue and making these compartments inaccessible for cells proteins and other higher molecular weight body substances and therefore minimise extensive oxidation damage reduces the accumulation of cells, fibrosis etc. and prolong the function of the sensor. By the method of the present invention the encapsulation process is substantially decreased which can be evidenced by the fact that the thickness of the fibrosis layer around the glucose measuring part of the sensor will be significant thinner when the sensor functions according to the method of the invention. Thus, in a histological section the thickness of the collagen capsule around the glucose measuring part of the sensor is less than 1 mm, such as less than 0.5 mm, preferably less than 0.1 mm, even more preferably less than 0.05 mm, most preferably less than 0.01 mm after a functional period, which is several days, one week, several weeks, several months, such as 3 months, preferably 6 months, most preferably one year as described in the following.

In order to increase the function of the glucose sensor in vivo and avoid e.g. the undesired calibration problems it is considered necessary to reduce the ROS, such as H₂O₂, to an amount, which is much lower than previously considered safe, i.e. considerably lower than 20 μM.

The present invention relates to use in a human of an implanted glucose oxidase based glucose sensor of a ROS removing compartment comprising catalase and/or a reactive oxygen species scavenger in order to reduce the diffusion of ROS, including H₂O₂, out of the sensor to a level where biointerference is substantially decreased or avoided in spite of the fact that the sensor is implanted in the human for a prolonged period of time.

The present invention thus provides a sensor for which the necessary amount of calibration is reduced when compared to a similar glucose sensor without a ROS removing compartment. Thus, by use of the method of the invention an implanted device will only necessitate calibration no more than once a day, such as once every second day, once every third day, or even only once a week for a period of time which is several days, one week, several weeks, several months, such as 3 months, preferably 6 months, most preferably one year.

In preferred embodiments of the invention the sensor is an implanted or semi-implanted sensor. Because of the decrease or avoidance of the biointerference, it is possible to have the implanted sensor function adequately several months, such as 3 months, preferably 6 months, most preferably one year. By the term “semi-implanted” is meant a sensor which is partly implanted but wherein part of the sensor is present outside the body. In practical terms a such sensor can be placed and removed by the person himself without the aid of medical personal. An example of a such sensor is a needle sensor produced e.g. by Minimed. By use of the method of the invention these sensors will function adequately for a long amount of time even if left within the body for at lest several days. Such semi-implanted sensors are thus in the present context within the concept of “implanted” sensors.

The important issue is that the level of ROS in the ROS removing compartment is to be considerably lower than the level of ROS, such as H₂O₂, naturally present in the particular body compartment so that no positive concentration gradient for H₂O₂ towards the sensor exists.

In preferred embodiments of the invention, the ROS removing compartment comprises catalase and/or one or more other reactive oxygen species scavengers. Examples of such reactive oxygen scavengers are polyphenols, such as as flavonoids, and plant phenolics, among them phenolic acids. The efficiency of phenolic compounds as anti-radicals and antioxidants is diverse and depends on many factors, such as the number of hydroxyl groups bonded to the aromatic ring, the site of bonding and mutual position of hydroxyls in the aromatic ring. Other examples of reactive oxygen scavengers are natural phenolic antioxidants (alpha-hydroxytyrosol, tyrosol, caffeic acid, alpha-tocopherol) as well as commercial phenolic antioxidants (BHT and BHA) and carotenoids.

In preferred embodiments the level of ROS, especially H₂O₂, immediately outside the glucose sensor is below 5 μM, such as below 3 μM, e.g. below 2 μM, preferably below 1 μM, more preferably below 0.5 μM, even more preferably below 0.3 μM, most preferably below 0.2 μM. In especially preferred embodiments the level of H₂O₂ immediately outside the glucose sensor is below 0.1 μM, such as below 0.05 μM, e.g. below 0.03 μM, preferably below 0.02 μM, more preferably below 0.01 μM, even more preferably below 0.00 μM, most preferably substantially 0 μM.

By use of the method according to the invention, the functional performance of the glucose sensor in vivo is improved. In particular the necessary amount of calibration is reduced as the reduced biointerference resulting from the reduced level/gradient of ROS, such as hydrogen peroxide, will increase the stability of the sensor over time, thereby minimising the number of re-calibrations of the sensor necessary for adequate performance over prolonged time periods. Presently, it is necessary to calibrate the commercially available sensors four times a day. By use of the method of the invention it is possible to prepare sensors which will only necessitate calibration no more than once a day, such as once every second day, once every third day, or even only once a week.

No such glucose sensors are presently available.

### LEGEND TO FIGURE

The invention is illustrated schematically in the figure which shows schematically the working electrode of a glucose sensor comprising the following compartments:

1. glucose oxidase producing H₂O₂ and an H₂O₂ detecting electrode

2. diffusion compartment through which glucose and O₂ diffuse to the glucose oxidase and the electrode and excess H₂O₂ may diffuse out to the body

3. ROS removing compartment e.g. a catalase membrane

4. Semipermeable compartment which hinders access of cells and high molecular compounds to the internal part of the electrode.

5. Biocompatible compartment

### EXAMPLES

Materials:

Electrochemical glucose needle sensors based on non-mediated glucose oxidase working electrodes in which no catalytic outer membrane is present. The needle sensors may be of either the two-electrode type (e.g. as described by Wilson. G. S. et al in U.S. Pat. No. 5,165,407) or three-electrode type.

The three-electrode type sensors are commercially available (e.g. MiniMed's continuous glucose sensor available from Medtronic MiniMed, 18000 Devonshire Street, Northridge, Calif. 91325-1219, USA) or homemade (e.g. as described by Ege, H. in WO 89/07139).

The sensors are powered by a potentiostat/galvanostat. Potentiostats suitable for different sensortypes are commercially available (e.g. uAutolab type II from Eco Chemie B. V., P.O. Box 85163, 3508 AD Utrecht, The Netherlands, or Amel instuments model 2059 from AMEL srl—Via S. Giovanni Battista de la Salle, 4, 20132 Milan—Italy).

Identical sensors are modified into two different groups C+ and C− by adding an extra outer membrane, where the sensors in the C+ group contains active hydrogen peroxide degrading catalyst (e.g. catalase) and the C− does not (e.g. heat inactivated catalyst or placebo catalytic inactive substance e.g. albumin). The extra outer membrane may be made on basis of Polyurethanes, alginates or other biocompatible material and a final biocompatible outermost membrane may also be added if suitable for in vivo function.

Phosphate Buffered Saline (PBS), or other standard physiological buffer is needed for the in vitro measurement. Known amounts of glucose are added to buffer samples until glucose concentrations (1 mM-30 mM range) relevant for in vivo measurements are reached. A small amount of preservative may be added (e.g. 1.2 mM sodium azide).

The glucose-PBS samples are also used for the initial equilibration (or “priming”) of the sensors until a stable electric current measurement (at an applied working electrode potential of 0.6 volt) is achieved (normally within about half an hour, if an initial potential of about 1.1 volt for a few minutes is applied to the working electrode).

Hydrogen peroxide liberated from the C+ or C− sensors can be measured by different commercially available peroxide test colour strip kits (MERCK EUROLAB A/S, Denmark) or by titration methods known to the person of ordinary skill in the art of analytical chemistry.

As the C+ or C− sensors are very small, the small amounts of hydrogen peroxide liberated is detected electrochemically by a hydrogen peroxide sensor probe. This probe is the end cross section of a thin (diameter 0.003″=0.08 mm) Teflon coated platinum wire from A-M Systems, Inc., PO Box 850, Carlsborg, Wash. 98324, USA, on which an electrode potential of 0.6 volt is applied relative to a reference electrode (e.g. Ag/AgCl homemade reference electrode or available from CH Instruments, Inc., 3700 Tennison Hill Drive, Austin, Tex. 78738, USA).

A normal platinum wire (Goodfellow Cambridge Limited, Ermine Business Park, HUNTINGDON, Cambridgeshire, PE29 6WR, England) is used as counter electrode and the electric current between the probe electrode and the counter electrode is measured by an potentiostat. As for the the glucose sensors an initial priming of the hydrogen peroxide sensor probe is done at a little higher potential. Samples of Glucose-PBS with a further addition of hydrogen peroxide to a hydrogen peroxide concentration in nanomolar to millimolar range are used for this priming. Such buffer samples are also used for establishment of calibration factors to be used in converting measured current to hydrogen peroxide concentration.

For some in vivo histological studies Identical sensors are modified into two other groups H− and H+ by heat inactivation (20-180 sec in room temperate water (H− group) or boiling water (H+ group)) without adding any further membrane.

Control of the heat inactivation of the hydrogen peroxide producing glucose oxidase in the H− group is done in vitro using the potentiostat. It is controlled that the H− does not respond to changes in glucose concentration, but still responds to changes in hydrogen peroxide concentrations. For this suitable samples of Glucose-PBS buffer with or without hydrogen peroxide (0.05-2 mM range) are used.

For some further in vivo histological studies of local effects of subcutaneous infusion of very small amounts of hydrogen peroxide, two separate pumps P− and P+ (type H-TRON plus V100 with connected infusion set Tender PT17/110 II from Disetronic Medical Systems, Inc., USA) are used. The P− pump is filled with standard physiological buffer with addition of hydrogen peroxide (less than 4% by volume). The P+ pump is filled with standard physiological buffer without addition of hydrogen peroxide. The pumps are then identically programmed to deliver over a few days very few micro liters for every 3-10 minutes.

### Experiment 1

In Vitro Characterisation:

A. The C+ and the C− sensors are connected to the potentiostats and equilibrated in PBS, to which a known amount of glucose has been added relevant for in vivo measurements. The time for reaching the initial stable current is noted (normally within half an hour) and also the response times to reach new stable plateau's of currents corresponding to various glucose concentrations are noted. Also and most important the differences in hydrogen peroxide liberated from the sensors into the buffer is detected. This can be done by measuring the hydrogen peroxide concentration gradients formed from the surface of the sensor out in the buffer. The gradients formed are detected by changing the distance between glucose sensor surface and the electrochemical hydrogen peroxide probe. This is done with either the glucose sensor or electrochemical hydrogen peroxide probe fixed to a measure table or micromanipulator with a micrometer scale. The distance of the probe from the sensor surface is incrementally reduced or increased and recorded together with the corresponding levels of measured current of the hydrogen peroxide probe.

B. Differences in hydrogen peroxide liberated from the sensors can also be measured in samples of glucose-PBS buffer after the glucose sensors has worked overnight in the buffer (preferably with a glucose concentration higher than 10 mM). This can be done by different commercially available peroxide test colour strip kits or by titration as described above.

C. An in vitro cell assay (e.g. as described in Callahan et al., 1990) using amount of killed cells due to liberation of hydrogen peroxide from the different groups of sensors can also be used in the characterisation.

### Experiment 2

After the difference is established in vitro between C+ and C−group sensors, the same sensors are tested for differences in vivo.

Suitable Laboratory Animals are Pigs or Dogs.

A. To show the C+group sensors advantages over the C−group, the sensors are implanted and the current is measured over some says (e.g. three days) together with blood sampling at some fixed time points (e.g. morning and evening). The blood samples are analysed for glucose concentration with standard methods (e.g. test strips and glucose meter in InDuo available from Novo Nordisk A/S, Denmark, or by use of laboratory instruments well known in standard clinical chemistry departments). From this the sensors+ performance are evaluated (precision, interval needed for calibration and lifetime). These performance characteristics may be supplemented with histological analysis of the resulting tissue around the implanted sensors with special emphasis on signs of killed cells and total amount of cells attracted to the sensors as well as signs of fibrosis, such as presence of collagen capsule around the glucose measuring part of the sensor.

B. To further support the histological analysis of experiment 2A the H− and H+ sensor group are also implanted in vivo for some days. At the end of the experiment, histological analysis of the tissue around the implanted sensors are conducted with special emphasis on signs of killed cells and total amount of cells attracted to the sensors as well as signs of fibrosis, such as presence of collagen capsule around the glucose measuring part of the sensor.

C. Also, to further support the histological analysis of experiment 2A, one animal is infused subcutaneously for a few days in vivo using both the P− and P+ pumps. Subsequently, the tissue at the site of infusion is analysed with special emphasis on signs of killed cells and total amount of cells attracted to the infusion sites as well as signs of fibrosis.

The extent of fibrosis can be evaluated using a standard techniques. The most common staining technique is known as Hematoxylin and Eosin (or H&E) staining. In order to stain the sections the wax needs to be removed. This is done using a wax solvent such as xylene. The slide is then hydrated using a series of descending alcohols (100%, 95%, 70%) and then water. The slide is then immersed in Hematoxylin stain, rinsed in running water (preferably alkaline), followed by staining with Eosin, and rinsing in water.

As an alternative to using H&E staining, the presence of collagen fibres can be determined using methods of histological staining known to a person with skills in the art. Examples of such stainings are the Van Giesen staining and the Masson Trichrome staining.

From the experiments it is clear that for optimal performance it is not enough to keep the amount of hydrogen peroxide liberated to the body lower than the level where cytotoxic (cell killing) effects are seen. In order to prevent cell attracting around the sensors, the concentration of the hydrogen peroxide hydrogen peroxide liberated to the body must be as low, or lower, as the concentration seen in the C+ group sensors.

### REFERENCES

Callahan, H. L.; Crouch, R. K.; James, E. R. Hydrogen Peroxide Is the Most Toxic Oxygen Species for Onchocerca Cervicalis Microfilariae. Parasitology 1990, 100 Pt 3, 407-415.

Halliwell, B.; Clement, M.; Long, L. Hydrogen peroxide in the human body. FEBS Letters 2000, 486, 10-13

Heller, A. Implanted Electrochemical Glucose Sensors for the Management of Diabetes. Annu. Rev. Biomed. Eng. 1999 01, 153-175

Jobst, G.; Moser, I.; Varahram, M.; Svasek, P.; Aschauer, E.; Trajanoski, Z.; Wach, P.; Kotanko, P.; Skrabal, F.; Urban, G. Thin-Film Microbiosensers for Glucose-Lactate Monitoring. Analytical Chemistry 1996, 68, 18, 3173-3179

Sauer, H.; Wartenberg, M.; Hescheler, J. Reactive Oxygen Species As Intracellular Messengers During Cell Growth and Differentiation. Cellular Physiology and Biochemistry 2001, 11, 173-186

Urban, G.; Jlobst., G.; Keplinger, F.; Aschauer, E.; Fasching, R.; Svasek, P. Miniturized integrated biosensors. Technology and Health Care, 1994, 1, 215-218

Urban, G.; Jobst, G.; Aschauer, E.; Tilado, O.; Svasek, P.; Varahram, M.; Ritter, Ch.; Riegebauer, J. Performance of integrated glucose and lactate thin-film microbiosensors for clinical analysers. Sensors and Actuators B, 1994, 18-19, 592-596

John Wiley & Son, “Biosensors in the body”, 1997, page 210

Zhang, J.; Johnston, G.; Stebler, B.; Keller, E. T. Hydrogen Peroxide Activates NF Kappa B and the Interleukin-6 Promoter Through NF Kappa B-Inducing Kinase. Antioxidants & Redox Signaling 2001, 3, 493-504

U.S. Ser. No. 2001/0,008,931

U.S. Pat. No. 5,165,407

WO 89/07139

WO94/10560

1. A method of improving the performance of a glucose oxidase based glucose sensor, said method comprising providing the glucose sensor with a ROS removing compartment capable of reducing the diffusion of ROS out of the glucose sensor to a level at which biointerference is abolished or substantially reduced.

2. A method according to claim 1, wherein the ROS removing compartment comprises catalase and/or one or more reactive oxygen species scavenger.

3. A method according to claims 1 or 2, wherein the reactive oxygen species is selected from the group consisting of H₂O₂, O.²⁻, and OH⁻.

4. A method according to any of the preceding claims, wherein the ROS removing compartment is able to ensure that the concentration of H₂O₂ in the tissue surrounding the glucose sensor remains below 10 μM.

5. A method according to claim 1 or 2, wherein substantially no activation of TGFO and substantially no monocyte chemotaxis occur in the tissue surrounding the glucose sensor.

6. A method according to any of the preceding claims, wherein the abolished or reduced biointerference leads to a decreased requirement for calibration of the glucose sensor when compared to the operation of a similar glucose sensor without a ROS removing compartment.

7. A method according to any of the preceding claims, wherein the sensor will require calibration no more than once a day, such as once every second day, once every third day, or once a week during functioning for a period of several days, one week, several weeks, several months, such as 3 months, preferably 6 months, most preferably one year.

8. A method according to any of the preceding claims, wherein the implanted sensor functions adequately several months, such as 3 months, preferably 6 months, most preferably one year.

9. A method according to any of the preceding claims, wherein the encapsulation process is substantially decreased as evidenced by the thickness of the collagen capsule around the glucose measuring part of the sensor being less than 1 mm, such as less than 0.5 mm, preferably less than 0.1 mm, even more preferably less than 0.05 mm, most preferably less than 0.01 mm after a functional period of time which is several days, one week, several weeks, several months, such as 3 months, preferably 6 months, most preferably one year.

10. A method according to any of the preceding claims wherein the glucose oxidase based glucose sensor is an implanted or semi-implanted glucose sensor.

11. Use of a ROS removing compartment in a glucose oxidase based glucose sensor so that biointerference is substantially decreased or avoided.

12. Use according to claim 9, wherein the ROS removing compartment comprises catalase and/or one or more reactive oxygen species scavengers.

13. Use according to any of claims 11 and 12, wherein the ROS removing compartment able to ensure that the concentration of H₂O₂ in the tissue surrounding the glucose sensor remains below 10 μM

14. Use according to any of claims 11 to 13, wherein the glucose sensor is implanted or semi-implanted in a human.

15. A glucose oxidase based glucose sensor comprising a ROS removing compartment capable of reducing the diffusion of ROS out of the glucose sensor to a level at which biointerference is abolished or substantially reduced

16. A glucose oxidase based glucose sensor according to claim 15, wherein the ROS removing compartment comprises catalase and/or one or more reactive oxygen species scavengers.

17. A glucose oxidase based glucose sensor according to claim 15 or 16 wherein the ROS removing compartment is able to ensure that the concentration of H₂O₂ in the tissue surrounding the glucose sensor remains below 10 μM

18. A glucose oxidase based glucose sensor according to any of claims 15 to 17, which is to be implanted or semi-implanted in a human.

19. A glucose oxidase based glucose sensor according to any of claims 15 to 18, wherein the ROS removing compartment is separated from the surrounding tissue by a biocompatible membrane.
